(19) United States
(12) Patent Application Publication
(10) Pub. No.: US 2018/0354001 A1
(43) Pub. Date: Dec. 13, 2018

(54) Cleaning Device for Cleaning a Connecting Aluminum Plate for Automobile Battery Integration and Cleaning Process Thereof

(71) Applicant: Shoukui Ye, Cixi City (CN)

(72) Inventors: Shoukui Ye, Cixi City (CN); Xiaohua Jiang, Cixi City (CN)

(21) Appl. No.: 15/739,527

(22) PCT Filed: Dec. 4, 2015
(86) PCT No.: PCT/CN2015/000846

§ 371 Date: Dec. 22, 2017

(30) Foreign Application Priority Data
Jun. 26, 2015 (CN) 201510364932.9

(51) Int. Cl. B08B 3/08; A61L 2/04; B08B 3/12; B08B 3/04

(52) U.S. Cl. CPC B08B 3/08; A61L 2/04; F26B 5/045; B08B 3/041; B08B 3/123

(57) ABSTRACT

A cleaning device for cleaning a connecting aluminum plate for automobile battery integration and cleaning process thereof, the cleaning device is disposed in a dust-free workshop, which comprises a transfer mechanism which successively transfers the connecting aluminum plate to different workshops; the workshops successively comprise three cleaning workshops, five rinsing workshops, a preheating workshop, a vacuum drying workshop, a baking and sterilizing workshop and an inspection and packaging workshop. The present invention can clean connecting aluminum plate in a pipelined manner and consume less energy, and by which the dyne level of the surface tension of the cleaned products can accord with the packaging requirement.

### RELATE APPLICATIONS

This application is a national phase entrance of and claims benefit to PCT Application for a probe for a cleaning device for cleaning a connecting aluminum plate for automobile battery integration and cleaning process thereof, PCT/CN2015/000846, filed on Dec. 4, 2015, which claims benefit to Chinese Patent Applications 201510364932.9, filed on Jun. 26, 2015. The specifications of both applications are incorporated here by this reference.

### FIELD OF THE INVENTION

The present invention relates to a cleaning device for cleaning a connecting aluminum plate for automobile battery integration and cleaning process thereof.

### DESCRIPTION OF THE PRIOR ART

With the continuous development of science and technology, the capacity of batteries is increasingly required in many fields comprising illumination, electronic products, vehicles and the like. Batteries are to be connected in series or in parallel to dispose the desired power supply volume and voltage requirement. Connection by using leads is complicated in operation and area-consuming. A connecting aluminum plate for automobile battery connecting integration emerges. Automobile batteries need to supply power to many parts of an automobile. Therefore, the connecting aluminum plate for automobile battery integration is a very important connection component in the present automobile power section. During the production and assembly of automobiles, connecting aluminum plate which have been cleaned and are impurity free will be used. The surface cleanliness of the connecting aluminum plate is the criteria of assessment of the connecting aluminum plate in many automobile manufacturers. In the prior art, there has been no systemic pipeline and cleaning process for cleaning a connecting aluminum plate.

### SUMMARY OF THE INVENTION

A first technical problem to be solved in the present invention is to provide a cleaning device for cleaning a connecting aluminum plate for automobile battery integration, which can clean connecting aluminum plate in a pipelined manner and consume less energy, and by which the dyne level of the surface tension of the cleaned products can accord with the packaging requirement.

A second technical problem to be solved in the present invention is to provide a cleaning process for cleaning a connecting aluminum plate.

To solve the first technical problem, the cleaning device for cleaning a connecting aluminum plate for automobile battery integration, which is disposed in a dust-free workshop, comprises a transfer mechanism which successively transfers the connecting aluminum plate to different workshops; the workshops successively comprise:

a first cleaning workshop, in which cleaning water with an alkali concentration of 2% to 3% and a temperature of 40° C. to 50° C. is filled, where the connecting aluminum plate are immerged into the cleaning water and cleaned by ultrasonic waves during the whole process;

a second cleaning workshop, in which cleaning water with an alkali concentration of 2% to 3% and a temperature of 40° C. to 50° C. is filled, where the connecting aluminum plate are immerged into the cleaning water and cleaned by ultrasonic waves during the whole process;

a third cleaning workshop, in which cleaning water with an alkali concentration of 2% to 3% and a temperature of 40° C. to 50° C. is filled, where the connecting aluminum plate are immerged into the cleaning water and cleaned by ultrasonic waves during the whole process;

a first rinsing workshop, in which deionized water with a temperature of 40° C. to 50° C. is filled, where the connecting aluminum plate are immerged into the deionized water and rinsed by ultrasonic waves during the whole process;

a second rinsing workshop, in which deionized water with a temperature of 40° C. to 50° C. is filled and a bubbler used for bubbling the deionized water is disposed, where the connecting aluminum plate are immerged into the deionized water;

a third rinsing workshop, in which deionized water with a temperature of 40° C. to 50° C. is filled, where the connecting aluminum plate are immerged into the deionized water and rinsed by ultrasonic waves during the whole process;

a fourth rinsing workshop, in which deionized water with a temperature of 40° C. to 50° C. is filled and a bubbler used for bubbling the deionized water is disposed, where the connecting aluminum plate are immerged into the deionized water;

a fifth rinsing workshop, in which deionized water with a temperature of 40° C. to 50° C. is filled, where the connecting aluminum plate are immerged into the deionized water and rinsed by ultrasonic waves during the whole process;

a preheating workshop, in which deionized water with a temperature of 60° C. to 70° C. is filled, where the connecting aluminum plate are immerged into the deionized water to be preheated;

a vacuum drying workshop, which is vacuumized and maintained under a vacuum pressure of −0.05 MPa to −0.15 MPa, where the moisture on the surfaces of the connecting aluminum plate is dried;

a baking and sterilizing workshop, which is with a temperature of 140° C. to 160° C., where the connecting aluminum plate is baked for 4 to 6 minutes; and

an inspection and packaging workshop, where the connecting aluminum plate after being baked and sterilized is inspected and packaged.

During the vacuumizing process in the vacuum drying workshop, the temperature inside the vacuum drying workshop will decrease. Therefore, as an improvement, the vacuum drying workshop is heated during the drying of the connecting aluminum plate in the vacuum drying workshop.

As another improvement, the transfer mechanism makes the connecting aluminum plate keeping in each of the first cleaning workshop, the second cleaning workshop, the third cleaning workshop, the first rinsing workshop, the second rinsing workshop, the third rinsing workshop, the fourth rinsing workshop and the fifth rinsing workshop for 3 to 5 minutes, keeping in the preheating workshop for 2 to 3 minutes, and keeping in the vacuum drying workshop for 4 to 5 minutes.

To solve the second technical problem, the process for cleaning a connecting aluminum plate is provided, wherein, the connecting aluminum plate is successively transferred to the following workshops from a dust-free workshop by a transfer mechanism:

a first cleaning workshop, in which cleaning water having an alkali concentration of 2% to 3% and a temperature of 40° C. to 50° C. is filled, where the connecting aluminum plate are immerged into the cleaning water and cleaned by ultrasonic waves during the whole process;

a second cleaning workshop, in which cleaning water having an alkali concentration of 2% to 3% and a temperature of 40° C. to 50° C. is filled, where the connecting aluminum plate are immerged into the cleaning water and cleaned by ultrasonic waves during the whole process;

a third cleaning workshop, in which cleaning water having an alkali concentration of 2% to 3% and a temperature of 40° C. to 50° C. is filled, where the connecting aluminum plate are immerged into the cleaning water and cleaned by ultrasonic waves during the whole process;

a first rinsing workshop, in which deionized water with a temperature of 40° C. to 50° C. is filled, where the connecting aluminum plate are immerged into the deionized water and rinsed by ultrasonic waves during the whole process;

a second rinsing workshop, in which deionized water with a temperature of 40° C. to 50° C. is filled and a bubbler used for bubbling the deionized water is disposed, where the connecting aluminum plate are immerged into the deionized water;

a third rinsing workshop, in which deionized water with a temperature of 40° C. to 50° C. is filled, where the connecting aluminum plate are immerged into the deionized water and rinsed by ultrasonic waves during the whole process;

a fourth rinsing workshop, in which deionized water with a temperature of 40° C. to 50° C. is filled and a bubbler used for bubbling the deionized water is disposed, where the connecting aluminum plate are immerged into the deionized water;

a fifth rinsing workshop, in which deionized water with a temperature of 40° C. to 50° C. is filled, where the connecting aluminum plate are immerged into the deionized water and rinsed by ultrasonic waves during the whole process;

a preheating workshop, in which deionized water with a temperature of 60° C. to 70° C. is filled, where the connecting aluminum plate are immerged into the deionized water to be preheated;

a vacuum drying workshop, which is vacuumized and maintained under a vacuum pressure of −0.05 MPa to −0.15 MPa, where the moisture on the surfaces of the connecting aluminum plate is dried;

a baking and sterilizing workshop, which is at a temperature of 140° C. to 160° C., where the connecting aluminum plate are baked for 4 to 6 minutes; and

an inspection and packaging workshop, where the connecting aluminum plate after being baked and sterilized is inspected and packaged.

As an improvement, the vacuum drying workshop is heated during the drying of the connecting aluminum plate in the vacuum drying workshop.

As another improvement, when the transfer mechanism makes the connecting aluminum plate keeping in each of the first cleaning workshop, the second cleaning workshop, the third cleaning workshop, the first rinsing workshop, the second rinsing workshop, the third rinsing workshop, the fourth rinsing workshop and the fifth rinsing workshop for 3 to 5 minutes, keeping in the preheating workshop for 2 to 3 minutes, and keeping in the vacuum drying workshop for 4 to 5 minutes.

Compared with the prior art, the present invention has the following advantages. By the cleaning device and cleaning process of the present invention, the dyne level of the surface tension (water) of the cleaned products is over 70% (for the application level, the dyne is at most 70; and the surface tension of water on objects at normal temperature is 72), which is far higher than the dyne value of 32 to 40 of American MIL-PRF-131K military-standard rust-proof, anti-static, shielding-effective, damp-proof aluminum foil packages, and the level of nonvolatile residues on the surfaces of the cleaned products reaches A/50 and the amount of residues is less than or equal to 0.02 NVR μg/cm2. The vacuumizing process at a normal temperature extracts water and undesired material from the surfaces of products, it is equivalent to perform one vacuum cleaning at a normal temperature of 5° C. and the connecting aluminum plate are then passed to the automatic baking tunnel at a high temperature of 140° C. to 160° C. to remove bacteria on the surfaces of products. The surface cleanliness of products subjected to the high-temperature processing reaches the highest level all over the world. Meanwhile, the cleaned products are vacuum dried and baked in the automatic baking tunnel, and this is the only one cleaning pipeline all over the world. The cleaning process of the present invention is energy-saving and the energy consumption is merely ½ of that in similar cleaning processes using hydrocarbons.

### BRIEF DESCRIPTION OF THE DRAWINGS

FIG. 1 is a structural diagram of a cleaning device for cleaning a connecting aluminum plate for automobile battery integration according to an Embodiment of the present invention.

### DETAILED DESCRIPTION OF THE PREFERRED EMBODIMENT

To enable a further understanding of the present invention content of the invention herein, refer to the detailed description of the invention and the accompanying drawings below:

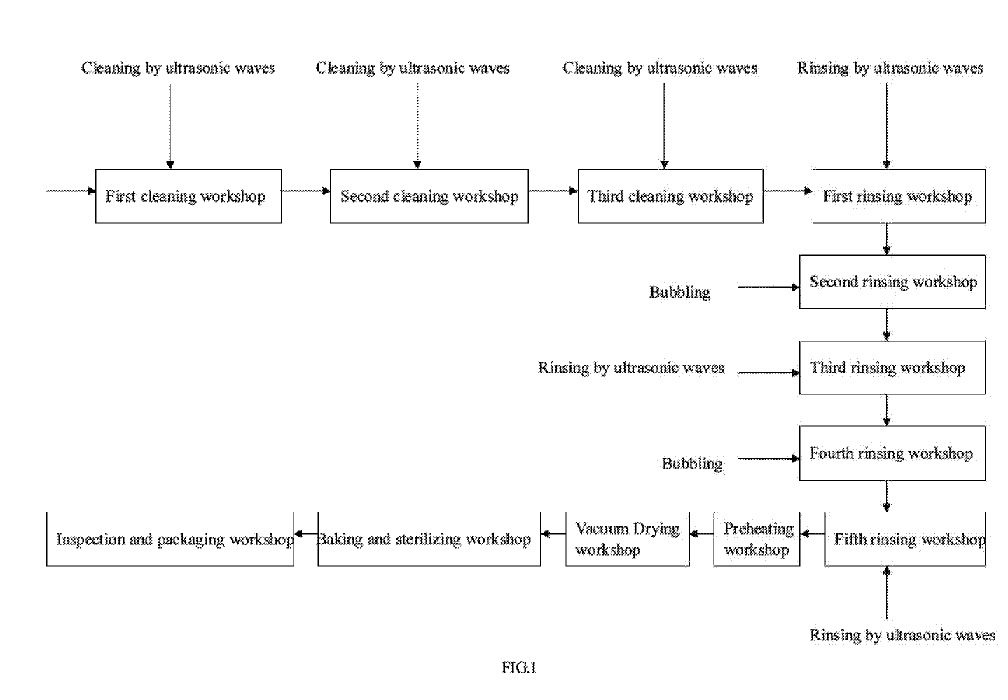

FIG. 1 shows a cleaning device for cleaning a connecting aluminum plate for automobile battery integration according to an embodiment the present invention, which is disposed in a dust-free workshop, comprising a transfer mechanism adopting existing conventional technology having a conveyor belt and a drive mechanism, which successively transfers the connecting aluminum plate to different workshops;

the workshops successively comprise:

a first cleaning workshop, in which cleaning water with an alkali concentration of 2% to 3% and a temperature of 40° C. to 50° C. is filled, where the connecting aluminum plate are immerged into the cleaning water and cleaned by ultrasonic waves during the whole process;

a second cleaning workshop, in which cleaning water with an alkali concentration of 2% to 3% and a temperature of 40° C. to 50° C. is filled, where the connecting aluminum plate are immerged into the cleaning water and cleaned by ultrasonic waves during the whole process;

a third cleaning workshop, in which cleaning water with an alkali concentration of 2% to 3% and a temperature of 40° C. to 50° C. is filled, where the connecting aluminum plate are immerged into the cleaning water and cleaned by ultrasonic waves during the whole process;

a first rinsing workshop, in which deionized water with a temperature of 40° C. to 50° C. is filled, where the connecting aluminum plate are immerged into the deionized water and rinsed by ultrasonic waves during the whole process;

a second rinsing workshop, in which deionized water with a temperature of 40° C. to 50° C. is filled and a bubbler used for bubbling the deionized water is disposed, where the connecting aluminum plate are immerged into the deionized water;

a third rinsing workshop, in which deionized water with a temperature of 40° C. to 50° C. is filled, where the connecting aluminum plate are immerged into the deionized water and rinsed by ultrasonic waves during the whole process;

a fourth rinsing workshop, in which deionized water with a temperature of 40° C. to 50° C. is filled and a bubbler used for bubbling the deionized water is disposed, where the connecting aluminum plate are immerged into the deionized water;

a fifth rinsing workshop, in which deionized water with a temperature of 40° C. to 50° C. is filled, where the connecting aluminum plate are immerged into the deionized water and rinsed by ultrasonic waves during the whole process;

a preheating workshop, in which deionized water with a temperature of 60° C. to 70° C. is filled, where the connecting aluminum plate are immerged into the deionized water to be preheated;

a vacuum drying workshop, which is vacuumized and maintained under a vacuum pressure of −0.05 MPa to −0.15 MPa, where the moisture on the surfaces of the connecting aluminum plate is dried;

a baking and sterilizing workshop, which is with a temperature of 140° C. to 160° C., where the connecting aluminum plate is baked for 4 to 6 minutes; and

an inspection and packaging workshop, where the connecting aluminum plate after being baked and sterilized is inspected and packaged.

The vacuum drying workshop is heated during the drying of the connecting aluminum plate in the vacuum drying workshop.

The transfer mechanism makes the connecting aluminum plate keeping in each of the first cleaning workshop, the second cleaning workshop, the third cleaning workshop, the first rinsing workshop, the second rinsing workshop, the third rinsing workshop, the fourth rinsing workshop and the fifth rinsing workshop for 3 to 5 minutes, keeping in the preheating workshop for 2 to 3 minutes, and keeping in the vacuum drying workshop for 4 to 5 minutes.

A cleaning process for cleaning the connecting aluminum plate above is: the connecting aluminum plate is successively transferred to the following workshops from a dust-free workshop by a transfer mechanism:

a first cleaning workshop, in which cleaning water having an alkali concentration of 2% to 3% and a temperature of 40° C. to 50° C. is filled, where the connecting aluminum plate are immerged into the cleaning water and cleaned by ultrasonic waves during the whole process;

a second cleaning workshop, in which cleaning water having an alkali concentration of 2% to 3% and a temperature of 40° C. to 50° C. is filled, where the connecting aluminum plate are immerged into the cleaning water and cleaned by ultrasonic waves during the whole process;

a third cleaning workshop, in which cleaning water having an alkali concentration of 2% to 3% and a temperature of 40° C. to 50° C. is filled, where the connecting aluminum plate are immerged into the cleaning water and cleaned by ultrasonic waves during the whole process;

a first rinsing workshop, in which deionized water with a temperature of 40° C. to 50° C. is filled, where the connecting aluminum plate are immerged into the deionized water and rinsed by ultrasonic waves during the whole process;

a second rinsing workshop, in which deionized water with a temperature of 40° C. to 50° C. is filled and a bubbler used for bubbling the deionized water is disposed, where the connecting aluminum plate are immerged into the deionized water;

a third rinsing workshop, in which deionized water with a temperature of 40° C. to 50° C. is filled, where the connecting aluminum plate are immerged into the deionized water and rinsed by ultrasonic waves during the whole process;

a fourth rinsing workshop, in which deionized water with a temperature of 40° C. to 50° C. is filled and a bubbler used for bubbling the deionized water is disposed, where the connecting aluminum plate are immerged into the deionized water;

a fifth rinsing workshop, in which deionized water with a temperature of 40° C. to 50° C. is filled, where the connecting aluminum plate are immerged into the deionized water and rinsed by ultrasonic waves during the whole process;

a preheating workshop, in which deionized water with a temperature of 60° C. to 70° C. is filled, where the connecting aluminum plate are immerged into the deionized water to be preheated;

a vacuum drying workshop, which is vacuumized and maintained under a vacuum pressure of −0.05 MPa to −0.15 MPa, where the moisture on the surfaces of the connecting aluminum plate is dried;

a baking and sterilizing workshop, which is at a temperature of 140° C. to 160° C., where the connecting aluminum plate are baked for 4 to 6 minutes; and

an inspection and packaging workshop, where the connecting aluminum plate after being baked and sterilized is inspected and packaged.

When the transfer mechanism makes the connecting aluminum plate keeping in each of the first cleaning workshop, the second cleaning workshop, the third cleaning workshop, the first rinsing workshop, the second rinsing workshop, the third rinsing workshop, the fourth rinsing workshop and the fifth rinsing workshop for 3 to 5 minutes, keeping in the preheating workshop for 2 to 3 minutes, and keeping in the vacuum drying workshop for 4 to 5 minutes.

1. A cleaning device for cleaning a connecting aluminum plate for automobile battery integration, which is disposed in a dust-free workshop, comprising a transfer mechanism which successively transfers the connecting aluminum plate to different workshops;
the workshops successively comprise:
a first cleaning workshop, in which cleaning water with an alkali concentration of 2% to 3% and a temperature of 40° C. to 50° C. is filled, where the connecting aluminum plate are immerged into the cleaning water and cleaned by ultrasonic waves during the whole process;
a second cleaning workshop, in which cleaning water with an alkali concentration of 2% to 3% and a temperature of 40° C. to 50° C. is filled, where the connecting aluminum plate are immerged into the cleaning water and cleaned by ultrasonic waves during the whole process;
a third cleaning workshop, in which cleaning water with an alkali concentration of 2% to 3% and a temperature of 40° C. to 50° C. is filled, where the connecting aluminum plate are immerged into the cleaning water and cleaned by ultrasonic waves during the whole process;
a first rinsing workshop, in which deionized water with a temperature of 40° C. to 50° C. is filled, where the connecting aluminum plate are immerged into the deionized water and rinsed by ultrasonic waves during the whole process;
a second rinsing workshop, in which deionized water with a temperature of 40° C. to 50° C. is filled and a bubbler used for bubbling the deionized water is disposed, where the connecting aluminum plate are immerged into the deionized water;
a third rinsing workshop, in which deionized water with a temperature of 40° C. to 50° C. is filled, where the connecting aluminum plate are immerged into the deionized water and rinsed by ultrasonic waves during the whole process;
a fourth rinsing workshop, in which deionized water with a temperature of 40° C. to 50° C. is filled and a bubbler used for bubbling the deionized water is disposed, where the connecting aluminum plate are immerged into the deionized water;
a fifth rinsing workshop, in which deionized water with a temperature of 40° C. to 50° C. is filled, where the connecting aluminum plate are immerged into the deionized water and rinsed by ultrasonic waves during the whole process;
a preheating workshop, in which deionized water with a temperature of 60° C. to 70° C. is filled, where the connecting aluminum plate are immerged into the deionized water to be preheated;
a vacuum drying workshop, which is vacuumized and maintained under a vacuum pressure of −0.05 MPa to −0.15 MPa, where the moisture on the surfaces of the connecting aluminum plate is dried;
a baking and sterilizing workshop, which is with a temperature of 140° C. to 160° C., where the connecting aluminum plate is baked for 4 to 6 minutes; and
an inspection and packaging workshop, where the connecting aluminum plate after being baked and sterilized is inspected and packaged.

2. The cleaning device of claim 1, wherein the vacuum drying workshop is heated during the drying of the connecting aluminum plate in the vacuum drying workshop.

3. The cleaning device of claim 1, wherein, the transfer mechanism makes the connecting aluminum plate keeping in each of the first cleaning workshop, the second cleaning workshop, the third cleaning workshop, the first rinsing workshop, the second rinsing workshop, the third rinsing workshop, the fourth rinsing workshop and the fifth rinsing workshop for 3 to 5 minutes, keeping in the preheating workshop for 2 to 3 minutes, and keeping in the vacuum drying workshop for 4 to 5 minutes.

4. A cleaning process for cleaning a connecting aluminum plate, wherein, the connecting aluminum plate is successively transferred to the following workshops from a dust-free workshop by a transfer mechanism:
a first cleaning workshop, in which cleaning water having an alkali concentration of 2% to 3% and a temperature of 40° C. to 50° C. is filled, where the connecting aluminum plate are immerged into the cleaning water and cleaned by ultrasonic waves during the whole process;
a second cleaning workshop, in which cleaning water having an alkali concentration of 2% to 3% and a temperature of 40° C. to 50° C. is filled, where the connecting aluminum plate are immerged into the cleaning water and cleaned by ultrasonic waves during the whole process;
a third cleaning workshop, in which cleaning water having an alkali concentration of 2% to 3% and a temperature of 40° C. to 50° C. is filled, where the connecting aluminum plate are immerged into the cleaning water and cleaned by ultrasonic waves during the whole process;
a first rinsing workshop, in which deionized water with a temperature of 40° C. to 50° C. is filled, where the connecting aluminum plate are immerged into the deionized water and rinsed by ultrasonic waves during the whole process;
a second rinsing workshop, in which deionized water with a temperature of 40° C. to 50° C. is filled and a bubbler used for bubbling the deionized water is disposed, where the connecting aluminum plate are immerged into the deionized water;
a third rinsing workshop, in which deionized water with a temperature of 40° C. to 50° C. is filled, where the connecting aluminum plate are immerged into the deionized water and rinsed by ultrasonic waves during the whole process;
a fourth rinsing workshop, in which deionized water with a temperature of 40° C. to 50° C. is filled and a bubbler used for bubbling the deionized water is disposed, where the connecting aluminum plate are immerged into the deionized water;
a fifth rinsing workshop, in which deionized water with a temperature of 40° C. to 50° C. is filled, where the connecting aluminum plate are immerged into the deionized water and rinsed by ultrasonic waves during the whole process;
a preheating workshop, in which deionized water with a temperature of 60° C. to 70° C. is filled, where the connecting aluminum plate are immerged into the deionized water to be preheated;
a vacuum drying workshop, which is vacuumized and maintained under a vacuum pressure of −0.05 MPa to −0.15 MPa, where the moisture on the surfaces of the connecting aluminum plate is dried;
a baking and sterilizing workshop, which is at a temperature of 140° C. to 160° C., where the connecting aluminum plate are baked for 4 to 6 minutes; and
an inspection and packaging workshop, where the connecting aluminum plate after being baked and sterilized is inspected and packaged.

5. The cleaning process of claim 4, wherein the vacuum drying workshop is heated during the drying of the connecting aluminum plate in the vacuum drying workshop.

6. The cleaning process of claim 4, wherein, when the transfer mechanism makes the connecting aluminum plate keeping in each of the first cleaning workshop, the second cleaning workshop, the third cleaning workshop, the first rinsing workshop, the second rinsing workshop, the third rinsing workshop, the fourth rinsing workshop and the fifth rinsing workshop for 3 to 5 minutes, keeping in the preheating workshop for 2 to 3 minutes, and keeping in the vacuum drying workshop for 4 to 5 minutes.
